(19) United States
(12) Patent Application Publication
(10) Pub. No.: US 2015/0196425 A1
(43) Pub. Date: Jul. 16, 2015

(54) OPHTHALMIC SURGICAL APPARATUS FOR PHACOEMULSIFICATION

(71) Applicant: Carl Zeiss Meditec AG, Jena (DE)

(72) Inventors: Christoph Kuebler, Oberkochen (DE); Michael Eichler, Aalen (DE)

(21) Appl. No.: 14/671,855

(22) Filed: Mar. 27, 2015

Related U.S. Application Data
(63) Continuation of application No. PCT/DE2013/000462, filed on Aug. 14, 2013.

(30) Foreign Application Priority Data
Sep. 27, 2012 (DE) 10 2012 018 983.4

(51) Int. Cl. A61F 9/007; A61M 1/00

(52) U.S. Cl. CPC A61F 9/00745; A61M 1/0001; A61M 1/0035; A61M 1/0076; A61M 1/0039; A61M 2210/0612

(57) ABSTRACT

An ophthalmic surgical apparatus for phacoemulsification includes an aspiration line to transport an aspiration fluid and particles of an eye generated by phacoemulsification. A suction vacuum pump draws in the aspiration fluid and the particles of the eye via the aspiration line and a flow limiter adjustably limits the flow to a flow lying in a range of 5 milliliter to 100 milliliter per minute. The aspiration line defines an inner cross section and has an outer wall. The flow limiter is arranged upstream of the suction vacuum pump and has at least two press-on elements. The elements are each configured to contact engage the outer wall so as to cause the inner cross section to be reduced. The press-on elements are disposed mutually spaced so as to define a predetermined distance therebetween and are arranged to be rotatably movable in a rotational direction about a common rotational axis.

### CROSS REFERENCE TO RELATED APPLICATIONS

This application is a continuation application of international patent application PCT/DE2013/000462, filed Aug. 14, 2013, designating the United States and claiming priority from German application 10 2012 018 983.4, filed Sep. 27, 2012, and the entire content of both applications is incorporated herein by reference.

### FIELD OF THE INVENTION

The invention relates to an ophthalmic surgical apparatus for phacoemulsification and an ophthalmic surgical system having such a device.

### BACKGROUND OF THE INVENTION

There are a number of surgical techniques for treating clouding within the eye lens, which is referred to as a cataract in medicine. The most common technique is phacoemulsification, in which a thin needle is introduced into the eye lens and excited to vibrate via ultrasound. The vibrating needle emulsifies the lens in its direct vicinity in such a way that the created lens particles can be suctioned away through a line via a pump. In the process, a rinsing fluid (irrigation fluid) is supplied, with the particles and the fluid being suctioned away through an aspiration line, which is usually arranged within the needle. Once the lens has been completely emulsified and removed, a new artificial lens can be inserted into the empty capsular bag, and so a patient treated thus can regain good visual acuity.

During the comminution of the eye lens via a needle vibrating with ultrasound, it is unavoidable during the surgical procedure that a relatively large particle comes to rest in front of the tip of the needle in such a way that this needle tip, or the suctioning opening thereof, is blocked. This state is referred to as occlusion. In such a case, a peristaltic pump, which is usually employed, builds up a suction pressure in the aspiration line that is many times higher than during occlusion-free operation. Additionally, there can be a greater energy influx for the movement of the needle such that the particle blocking the needle is comminuted. Alternatively, a reversal of the operating direction of the peristaltic pump can also remove the particle from the needle tip again, such that there can once again be conventional suctioning away of the fluid and the small particles. Consequently, an occlusion is broken up at such a moment, with the previously applied large negative pressure being reduced very quickly. The suction generated thereby may lead not only to small particles and fluid being pulled to the aspiration needle, but also to part of the capsular bag coming into contact with the needle. If the capsular bag is pierced, this leads to significant complications for the patient, which must be avoided at all costs.

The peristaltic pump could be operated in such a way that, for example, it suctions away aspiration fluid and particles at a high number of revolutions so that a relatively high negative pressure is generated in the aspiration line. Although this would achieve faster emulsification of the lens, there would then be a significant drop in intraocular pressure. A low intraocular pressure is dangerous since the eye can easily collapse in the case of low pressure and, for example, the pupil can be excited to a clearly perceivable vibration. If, moreover, an occlusion and subsequent breakup should occur during such a method of operation, large variations in the intraocular pressure would occur, increasing the risk of negative pressure being generated in the eye, the negative pressure allowing the eye to collapse. The reason for this lies in the operation of the peristaltic pump. The peristaltic pump must be switched off at the beginning of an occlusion and switched back on again after the occlusion is broken up. After the occlusion is broken up, approximately 30 ms pass before the pump can convey its normal volume flow again, during which period of time a significant variation in the intraocular pressure cannot be prevented.

United States patent application publication 2008/0125698 A1 describes a system and a device for controlling energy and flow speed. To this end, provision is made for a suction pump, via which fluid can be suctioned out of an aspiration line, wherein a flow limiter can act on the outer wall of the aspiration line such that it is possible to reduce the effective resistance of the flow path in the aspiration line.

### SUMMARY OF THE INVENTION

It is an object of the invention to develop an ophthalmic surgical apparatus for phacoemulsification, which enables a relatively short operation duration, wherein, nevertheless, there is only a relatively small reduction in the intraocular pressure compared to the normal interior pressure of an eye. Additionally, there should not be large variations in the intraocular pressure when an occlusion is broken up, wherein this should also be possible when relatively large lens particles are suctioned away. Furthermore, the outlay for controlling the employed pump should remain low. It is moreover an object of the invention to develop an ophthalmic surgical system having such an ophthalmic surgical apparatus.

For the ophthalmic surgical apparatus, the object is achieved by an ophthalmic surgical apparatus for phacoemulsification having:
an aspiration line which is suitable for transporting an aspiration fluid and particles of an eye lens generated by phacoemulsification;
a suction vacuum pump, with which the aspiration fluid and the particles can be suctioned by means of the aspiration line; and,
a flow limiter, which limits the flow in the aspiration line, wherein the flow can be set to a value in the range from 5 milliliters per minute to 100 milliliters per minute, and the flow limiter is arranged upstream of the suction vacuum pump when viewed in the flow direction,
wherein the flow limiter has at least two press-on elements, which are respectively positionable on an aspiration line outer wall in such a way that the aspiration line internal cross section is reduced, wherein the press-on elements are arranged at a predetermined distance from one another and in a manner movable about a common rotational axis in the direction of rotation.

Using the suction vacuum pump, it is possible to achieve a relatively high negative pressure in the aspiration line during the whole duration of the phacoemulsification. The suction vacuum pump is preferably dimensioned in such a way that the negative pressure in the aspiration line is at least 400 mmHg. So that the intraocular pressure does not drop to a dangerously low level at this high suction pressure, the ophthalmic surgical device has a flow limiter which restricts the flow in the aspiration line to a value in the range from 5 milliliters per minute to 100 milliliters per minute. In the case of the low value of 5 milliliters per minute, the intraocular pressure practically does not drop during phacoemulsification. At the upper limit of 100 milliliters per minute, the intraocular pressure reduces slightly but still remains so stable that there is no risk of the eye collapsing. This also leads to the volume flow in the irrigation line being able to be kept relatively low.

If an occlusion occurs in the aspiration line such that the aspiration line is blocked, the suction pressure in the aspiration line increases to approximately 600 mmHg, meaning the maximum suction power of a suction vacuum pump. When the occlusion breaks up, the pressure in the aspiration line of 600 mmHg immediately drops to, for example, 400 mmHg, and so no strong pressure variations are induced in the aspiration line. This leads to the intraocular pressure likewise not being subject to strong pressure variations, and so the dangerous situation in which the capsular bag is drawn toward a needle of the handpiece does not occur. The use of a suction vacuum pump is advantageous since it need not be switched off or on during occlusion-free operation, during an occlusion and after an occlusion has been broken up. In contrast to a peristaltic pump, the suction vacuum pump suctions without interruptions, and so there is no control outlay depending on an occlusion for the suction vacuum pump. Therefore, the problem addressed is solved completely by the ophthalmic surgical device according to the invention.

In accordance with the invention, the flow limiter has at least two press-on elements, which are positionable on an aspiration line outer wall in such a way that the aspiration line internal cross section is reduced. In such an arrangement, the press-on element has no contact with an aspiration fluid to be transported, and so completely sterile suctioning away still is possible.

In accordance with the invention, the at least two press-on elements are, additionally, arranged at a predetermined distance from one another and in a manner movable about a common rotational axis in the direction of rotation. As a result of two press-on elements being arranged at a distance from one another, it is possible for the aspiration line not to be reduced in terms of its internal cross section in this distance range. What this achieves is that relatively large lens particles can collect in this distance range, the relatively large lens particles being conveyed along the aspiration line by the rotational movement of the press-on elements. Although a press-on element is positionable on the aspiration line outer wall in such a way that the aspiration line internal cross section is reduced, and so relatively large lens particles can no longer pass through this constriction, a relatively large lens particle can be conveyed along the aspiration line as a result of the distance of two press-on elements from one another and a rotation of the press-on elements about a common axis.

Preferably, an aspiration fluid container for holding the aspiration fluid and the particles is arranged between the suction vacuum pump and the flow limiter. Such a container brings about dampening of possibly occurring vibrations in the aspiration line. As the ratio between unfilled container volume and container volume filled with aspiration fluid increases, the damping becomes stronger.

The press-on elements can be provided in such a way that these press on the aspiration line outer wall in such a way that the extent of the aspiration line internal cross section has a value in the range from 0.02 mm to 0.3 mm. If the extent of the aspiration line internal cross section only has a value of 0.02 mm, very strong flow limiting is possible therewith, and so the flow can be approximately 5 milliliters per minute. The flow in the aspiration line increases with increasing extent of the aspiration line internal cross section, wherein the maximum flow of 100 milliliters per minute can be obtained at an extent of approximately 0.3 mm. In general, a press-on element brings about a strong reduction in the flow in the aspiration line by such a reduction in the aspiration line internal cross section.

In a preferred embodiment, the press-on elements can be part of a peristaltic pump. A device for phacoemulsification often has a peristaltic pump, and so the flow limiter need not be an additional separate component but can be formed by the peristaltic pump.

In accordance with one embodiment of the invention, the press-on elements of the peristaltic pump are movable along a normal with respect to the longitudinal axis of the aspiration line. This enables a very simple reduction in the aspiration line internal cross section.

The suction vacuum pump can be a venturi pump or a centrifugal pump or a turbine pump. Using such pumps, it is possible to obtain a relatively high suction pressure in the aspiration line during the whole duration of the phacoemulsification.

For the ophthalmic surgical system, the object is achieved by an ophthalmic surgical system having an ophthalmic surgical apparatus for phacoemulsification having an aspiration line configured to transport an aspiration fluid and particles of an eye generated by phacoemulsification in a predetermined flow direction; the ophthalmic surgical apparatus further having a suction vacuum pump and a flow limiter; the suction vacuum pump being configured to draw in the aspiration fluid and the particles of the eye via the aspiration line; the flow limiter being configured to adjustably limit the flow to a flow lying in a range of 5 milliliter to 100 milliliter per minute; the aspiration line defining an inner cross section thereof and having an outer wall; the flow limiter being arranged ahead of the suction vacuum pump with respect to the flow direction; the flow limiter having at least two press-on elements; the press-on elements each being configured to contact engage the outer wall so as to cause the inner cross section to be reduced; the press-on elements being disposed in spaced relationship to each other so as to define a predetermined distance therebetween and being arranged to be rotatably movable in a rotational direction about a common rotational axis; a control unit configured to control the suction vacuum pump; and, a phacoemulsification handpiece including a portion of the aspiration line.

### BRIEF DESCRIPTION OF THE DRAWINGS

The invention will now be described with reference to the drawings wherein:

FIG. 1 is a schematic of an ophthalmic surgical device according to the invention;

FIG. 2 is a schematic of signal profiles of an intraocular pressure, a suction pressure in an aspiration line and a volume flow in an irrigation line as a function of time;

FIG. 3 is a schematic of a flow limiter interacting with an aspiration line;

FIG. 4 shows a detail from FIG. 3 with a press-on element which interacts with the aspiration line;

FIG. 5 shows a cross-sectional view along the cut line A-A in FIG. 4; and,

FIG. 6 shows a cross-sectional view along the cut line B-B in FIG. 4.

### DESCRIPTION OF THE PREFERRED EMBODIMENTS OF THE INVENTION

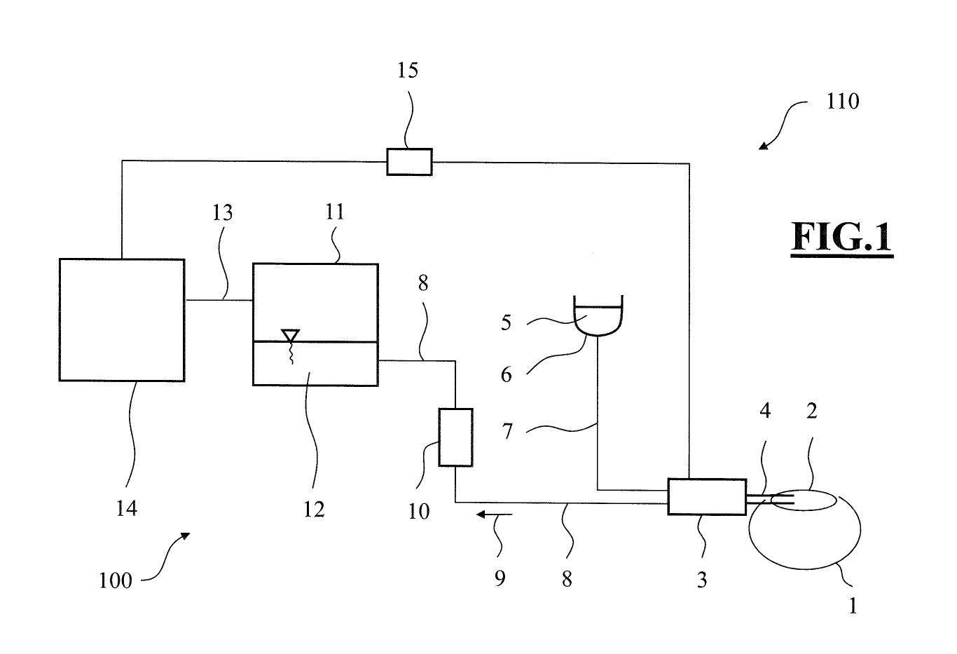

FIG. 1 is a schematic of an ophthalmic surgical system 110 with an embodiment of an ophthalmic surgical apparatus 100. An eye 1 with a lens 2 is treated via a phacoemulsification handpiece and a needle 4 fastened thereto, the latter moving longitudinally with ultrasonic vibrations. An irrigation fluid 5 in an irrigation fluid container 6 is led to the phacoemulsification handpiece 3 via an irrigation fluid line 7 such that irrigation fluid 5 can emerge in the region of the distal end of the needle 4. The lens particles generated by the vibrating needle 4 are transported away, together with the fluid, through an aspiration line 8 in the suction direction (see arrow 9). Here, they reach a flow limiter 10, pass the latter and are conveyed further along the aspiration line 8 as far as an aspiration fluid container 11. The lens particles and the aspiration fluid, which together are denoted by the reference numeral 12, can collect in the container 11. The container 11 is connected via a suction line 13 to a suction vacuum pump 14, which builds up suction pressure in the suction line 13, the aspiration fluid container 11, the aspiration line 8 and the flow limiter 10 up to the distal end of the needle 4. The suction vacuum pump 14 is connected to a control unit 15, wherein the control unit 15 is additionally connected to the handpiece 3 in order, for example, to control the energy supply for the longitudinal movement of the needle 4.

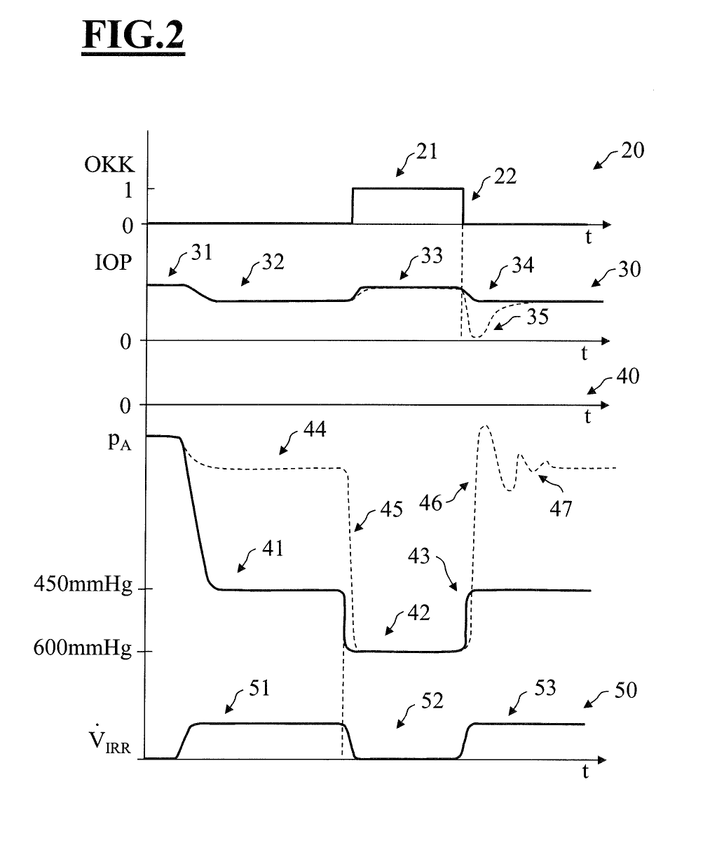

FIG. 2 shows a schematic illustration of signal profiles of the intraocular pressure IOP, of the negative pressure p_{A} in the aspiration line and of the volume flow V_{IRR} in the irrigation line as a function of time. It is assumed that the intraocular pressure has a predetermined value prior to a phacoemulsification, see reference sign 31 in diagram 30. The intraocular pressure reduces insignificantly at the start of the phacoemulsification, see reference sign 32, wherein the intraocular pressure remains constant throughout the whole phacoemulsification provided that there is no occlusion in the aspiration line 8. Diagram 40 shows that the negative pressure in the aspiration line increases to a relatively high level at the start of the phacoemulsification, in this case 450 mmHg, and remains constant there, see reference sign 41. The volume flow of the irrigation line likewise increases to a constant level at the start of the phacoemulsification, see reference sign 51 in diagram 50. If the aspiration line is blocked such that an occlusion occurs, see reference sign 21 in diagram 20, the intraocular pressure increases again to normal pressure as a result of the lack of suctioning away of the aspiration fluid and the particles in the eye, see reference sign 33, wherein, at the same time, the negative pressure in the aspiration line increases to the maximum value, for example, 600 mmHg, see reference sign 42. Since no fluid can be suctioned through the aspiration line, the volume flow through the irrigation line is reduced, see reference sign 52 in diagram 50.

If there is a breakthrough in the occlusion, see reference sign 22 in diagram 20, the intraocular pressure falls slightly again, see reference sign 34 in diagram 30, wherein the negative pressure in the aspiration line likewise sinks slightly in a very quick and almost delay-free manner, see the full line denoted by reference sign 43 in diagram 40. As a result of the restarting aspiration of lenses and the fluid, the volume flow through the irrigation line is once again increased to the value predetermined previously, see reference sign 53 in diagram 50.

In diagram 30, a dashed line depicts how this situation behaves in respect of the intraocular pressure at the beginning of an occlusion and at the breakthrough of an occlusion if, instead of the suction vacuum pump according to the invention for suctioning away the aspiration fluid and the lens particles, a peristaltic pump in accordance with the prior art is used. It can clearly be identified—see reference sign 35 in diagram 30—that the intraocular pressure after the breakthrough of an occlusion may sink down to almost 0 mmHg or even to less than 0 mmHg, and so the dangerous situation of the eye collapsing may occur here.

With a dashed line, diagram 40 shows the associated negative pressure in the aspiration line if the aspiration is carried out via a peristaltic pump in accordance with the prior art. During normal phacoemulsification, the negative pressure in the aspiration line is relatively low, see reference sign 44 in diagram 40, but it increases very strongly when an occlusion occurs, see reference sign 45 in diagram 40. The negative pressure falls very strongly after the breakthrough of an occlusion, see reference sign 46 in diagram 40, and for a while undergoes post-pulse oscillations, see reference sign 47 in diagram 40. These strong vibrations become noticeable in the profile of the intraocular pressure, see reference sign 35 in diagram 30.

Therefore, the ophthalmic surgical apparatus 100 according to the invention allows constant suction with relatively high negative pressure in the aspiration line, see reference signs 41 and 42 in diagram 40, wherein there are no strong pressure variations in the aspiration line and in the eye chamber after a breakthrough of an occlusion, see reference sign 43 in diagram 40 and reference sign 34 in diagram 30. It is therefore possible within a relatively short period of time to carry out a phacoemulsification in a very reliable manner and with only little risk.

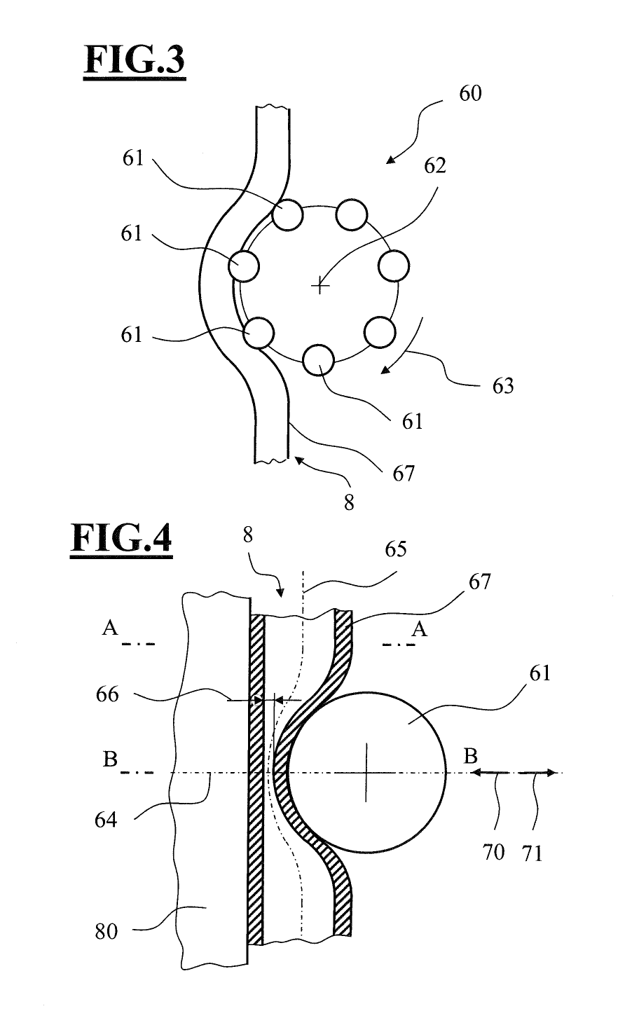

FIG. 3 shows an aspiration line 8 and a flow limiter in the form of a peristaltic pump 60. The peristaltic pump 60 has press-on elements 61, which are movable in a rotational direction 63 around an axis of rotation 62. The aspiration line 8 can extend in such a way that the press-on elements 61 are pressed on an aspiration line outer wall 67 of the aspiration line 8.

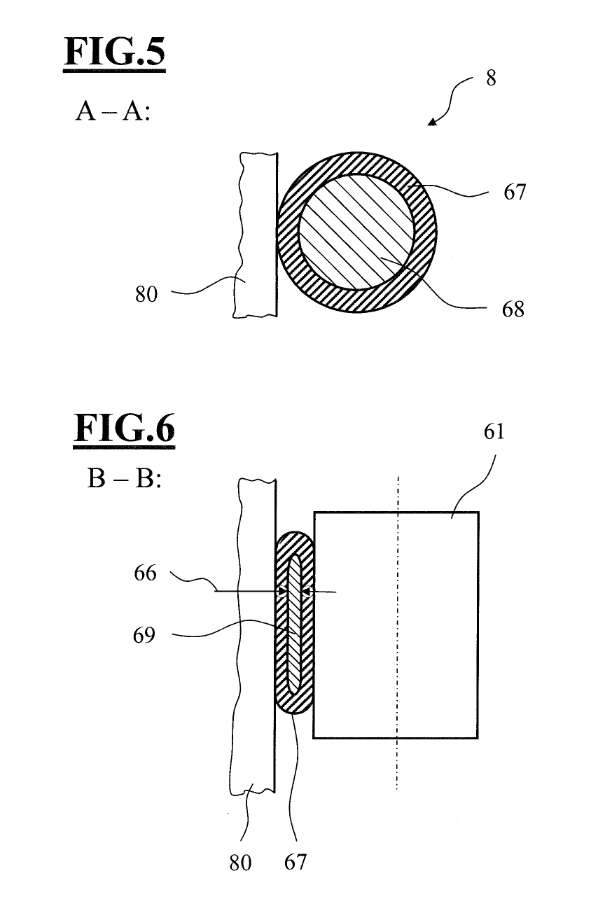

FIG. 4 shows a detail of such a situation, in which a press-on element 61 presses on an aspiration line outer wall 67 of the aspiration line 8 which, on the side opposite thereto, rests against a rest 80. The press-on element 61 can be moved along a normal 64 with respect to the longitudinal axis 65 of the aspiration line 8. An extent 66 of an aspiration line internal cross section emerges dependent on the set position of the press-on element 61, and so the flow through the aspiration line 8 can be restricted. Without the action of a press-on element 61, see section A-A in FIG. 4, the aspiration line 8 has an aspiration line internal cross section 68, see FIG. 5. A section along the cut line B-B in FIG. 4 results in an aspiration line internal cross section 69 with an extent 66, wherein the aspiration line internal cross section 69 is significantly smaller than the aspiration line internal cross section 68. Therefore, it is possible via the lateral movement of the press-on element 61 in the line of the normal 64 along the arrows 70 or 71 to reduce the aspiration line internal cross section 68 to an aspiration line internal cross section 69.

It is understood that the foregoing description is that of the preferred embodiments of the invention and that various changes and modifications may be made thereto without departing from the spirit and scope of the invention as defined in the appended claims.

What is claimed is:

1. An ophthalmic surgical apparatus for phacoemulsification comprising:
an aspiration line configured to transport an aspiration fluid and particles of an eye generated by phacoemulsification in a predetermined flow direction;
a suction vacuum pump configured to draw in the aspiration fluid and the particles of the eye via said aspiration line;
a flow limiter configured to adjustably limit said flow to a flow lying in a range of 5 milliliter to 100 milliliter per minute;
said aspiration line defining an inner cross section thereof and having an outer wall;
said flow limiter being arranged ahead of said suction vacuum pump with respect to said flow direction;
said flow limiter having at least two press-on elements;
said elements each being configured to contact engage said outer wall so as to cause said inner cross section to be reduced; and,
said press-on elements being disposed in spaced relationship to each other so as to define a predetermined distance therebetween and being arranged to be rotatably movable in a rotational direction about a common rotational axis.

2. The apparatus of claim 1 further comprising an aspiration fluid container arranged between said suction vacuum pump and said flow limiter and configured to receive the aspiration fluid and the particles of the eye.

3. The apparatus of claim 2, wherein:
said inner cross section defines a height;
said press-on elements are configured to press on said outer wall so as to cause said height to lie in a range of 0.02 mm to 0.3 mm.

4. The apparatus of claim 1 further comprising:
a peristaltic pump; and,
said press-on elements being components of said peristaltic pump.

5. The apparatus of claim 4, wherein:
said aspiration line defines a longitudinal axis;
said press-on elements of said peristaltic pump are movable along a normal with respect to said longitudinal axis.

6. The apparatus of claim 1, wherein said suction vacuum pump is one of a venturi pump, a centrifugal pump and a turbine pump.

7. A ophthalmic surgical system comprising:
an ophthalmic surgical apparatus for phacoemulsification having an aspiration line configured to transport an aspiration fluid and particles of an eye generated by phacoemulsification in a predetermined flow direction;
said ophthalmic surgical apparatus further having a suction vacuum pump and a flow limiter;
said suction vacuum pump being configured to draw in the aspiration fluid and the particles of the eye via said aspiration line;
said flow limiter being configured to adjustably limit said flow to a flow lying in a range of 5 milliliter to 100 milliliter per minute;
said aspiration line defining an inner cross section thereof and having an outer wall;
said flow limiter being arranged ahead of said suction vacuum pump with respect to said flow direction;
said flow limiter having at least two press-on elements;
said press-on elements each being configured to contact engage said outer wall so as to cause said inner cross section to be reduced;
said press-on elements being disposed in spaced relationship to each other so as to define a predetermined distance therebetween and being arranged to be rotatably movable in a rotational direction about a common rotational axis;
a control unit configured to control said suction vacuum pump; and,
a phacoemulsification handpiece including a portion of said aspiration line.
